(19) United States
(12) Patent Application Publication
(10) Pub. No.: US 2007/0162310 A1
(43) Pub. Date: Jul. 12, 2007

(54) Medical office electronic management system and method

(75) Inventor: Timothy W. Schmidt, Pittsford, NY (US)

Correspondence Address: SIMPSON & SIMPSON, PLLC, 5555 MAIN STREET, WILLIAMSVILLE, NY, 14221-5406, US

(73) Assignee: Health Systems Technology, Inc., Rochester, NY (US)

(21) Appl. No.: 11/323,956

(22) Filed: Dec. 30, 2005

(51) Int. Cl. G06F 19/00; A61B 5/00

(52) U.S. Cl. 705/3; 600/300

(57) ABSTRACT

The invention broadly comprises a method for processing work flow regarding a medical visit including the steps identifying the selection, in an electronic medical records system (EMRS), of a condition during the visit and displaying a treatment plan regarding the condition. In some aspects, the selection is regarding treatment, documentation, or billing. In some aspects, the method at least partially populates a library from a first standard of treatment or with data from the EMRS. In some aspects, the method accepts an editing input and modifies the treatment plan in response to the editing input. In some aspects, the treatment plan designates a guideline as a default guideline. In some aspects, the method generates at least one code set value regarding the condition and communicates the at least one code set value to the EMRS for use in prepopulating at least one data entry form.

### FIELD OF THE INVENTION

The invention relates generally to management of electronic medical records. In particular, the invention relates to managing records associated with a visit to a medical practitioner. Further, the invention relates to a trainable management system and method for automatically updating medical records regarding a visit to a medical practitioner.

### BACKGROUND OF THE INVENTION

Medical record-keeping, including storage, retrieval, analysis, and transmittal of patient records, as well as office management, including scheduling and billing, are vital aspects of medical office management. Typically, however, these functions are performed by separate systems that are not integrated together. For example, in regard to general workflow, usually, when a patient checks in, a nurse or assistant periodically checks a sign-in sheet or with the front desk to determine that a patient has arrived. After placing the patient in a room and recording vitals in the patient's chart, the nurse or assistant typically uses colored flags outside the door to indicate that a patient is there, and the doctor periodically checks the colored flags to know that a patient is waiting. If the doctor needs nurse intervention for a test, injection, handout, etc., the doctor walks to the nurse's area and communicates verbally. The nurse or assistant then does what was requested, and then finds the doctor to communicate that the requested task is completed. The doctor may or may not return to the patient. The doctor then writes down on paper instructions for the front desk, upon the patient's check-out.

Published United States Patent Application No. 2004/0088192, incorporated herein by reference, discloses an electronic medical records and medical office management system (EMRS) including electronic storage, retrieval, analysis, and transmittal of patient records. In some aspects, the EMRS includes an electronic encounter feature, that integrates with billing, generates referrals, orders labs and tests, allows for medications to be prescribed, and directs for follow-up visits, as needed. The system also includes automatic electronic notification, integrated appointment scheduling, preventive care profile integration with the electronic encounter, and history and exam findings entry, which converts data to text. Note creation draws from a patient record, encounter data and events triggered, and patient history and exam findings.

The following discussion is regarding a typical prior art procedure for conducting a medical office visit using a prior art EMRS, for example, the system disclosed in US 2004/0088192. It should be understood that the following is a general discussion and is not meant to cover the entire range of possibilities regarding prior art methods of conducting medical office visits, such as using a prior art EMRS. A visit begins with a medical practioner (MP), such as a medical doctor, nurse practitioner, or physician's assistant, identifying a problem or complaint. In the case of a visit for an acute problem this process begins by the patient describing a symptom, e.g., “I've had this pain in my lower back whenever I . . . ” The process continues when the MP collects information by asking specific questions about the symptom in order to make a diagnosis. In the case of a follow-up visit, the patient presents for evaluation and treatment of a problem that has been previously diagnosed. In both cases, the MP obtains and documents a history on the problem or complaint. The documentation of this information constitutes the subjective part of his visit note.

EMRSs have various methods of capturing this information. The most fundamental of them is to simply type the subjective information into a text box (not shown) presented as part of a data entry form. The following example is based a fictitious patient X, presenting for a follow-up visit for Hypercholesterolemia. History forms (not shown) are grouped into categories that allow for pertinent information to be entered, for example, symptoms, dietary compliance etc. Because this patient is being seen for Hypercholesterolemia, a Cardiac Follow-Up form (not shown) is selected. After obtaining a history from a patient, an MP will typically examine the patient and record her findings in the second part of the note, the objective portion. The areas the MP examines, of course, depend on the problem or complaint for which he is seeing the patient. Although EMRS programs capture this information differently, in general, the program displays a series of forms in which the MP makes entries, for example, recording general appearance, weight, etc. There may be forms for general findings and forms for specific areas, such as the heart, chest, extremities etc. The data entered in the above forms is translated by the system into text as part of the objective portion of the current visit note. Further, the above data is collected as part of the examination for inclusion in the note on a patient being seen for follow-up on Hypercholesterolemia.

After the MP has interviewed and examined the patient, she is ready to render her assessment of the patient's problem or complaint and determine the plan for treatment and/or further evaluation. Traditionally, the documentation of these components of the visit occurs in the Assessment and Plan section of the doctor's note. In the present example, the plan for the patient includes:
1. An EKG to determine if the patient has suffered any heart damage from the Hypercholesterolemia.
2. A fasting lipid profile to evaluate the current cholesterol level.
3. Provision of a Cholesterol Lowering Diet handout.

To order an EKG, the MP opens a new form (not shown) and selects the appropriate check boxes. In like manner, the MP opens and fills out separate forms (not shown) for lab work and handouts. Upon completion of the visit, a note is prepared, summarizing the visit. For each issue that a patient may present, the MP goes through a similar procedure of opening multiple forms. For example, if the fictional patient should also present for Diabetes, the MP would process a series of screens and forms regarding Diabetes. Unfortunately, known EMRS require the MP to search for, select, and fill out large numbers of screens and data forms and do not coordinate the entry of data among the screens and data forms. In particular, known EMRSs do not present a condensed presentation of appropriate guidelines for treating a known problem.

Thus, there is a long-felt need to provide a computer-based method and system for presenting an MP with known or suggested treatment actions and options regarding the conditions.

### SUMMARY OF THE INVENTION

The invention broadly comprises a method for processing work flow regarding a medical visit including the steps identifying the selection, in an electronic medical records system (EMRS), of a condition during the visit and displaying a treatment plan regarding the condition, where the steps of identifying and displaying, and the remaining steps of the method, are performed by at least one specially programmed general-purpose computer. In some aspects, the selection is regarding treatment, documentation, or billing. The method selects the treatment plan from a library comprising a plurality of treatment plans. In some aspects, the method at least partially populates the library from a first standard of treatment or the library accepts data from the EMRS and at least partially populates the library with the data. In some aspects, the method accepts an editing input and modifies the treatment plan in response to the editing input.

In some aspects, the treatment plan includes a first guideline and the method designates the first guideline as a default guideline. In some aspects, the method accesses a second standard of treatment or accepts an input from a user, the input regarding the first guideline. Then, the method designates the first guideline as a default guideline in accord with the input or in accord with the second standard of treatment. In some aspects, the treatment plan includes a second guideline and the second guideline is selected from the group consisting of procedure tasks, medication tasks, written materials tasks, documentation tasks, and practioner information tasks. In some aspects, the treatment plan includes a third guideline and the method executes the third guideline. In some aspects, the third guideline is selected from the group consisting of sending information to other parties; generating medication prescriptions; creating letters regarding the patient; providing written materials for the patient; generating medical service orders; triggering procedures to be performed; and generating a history and documenting examination findings.

In some aspects, the EMRS includes at least one data entry form and the method generates at least one code set value regarding the condition and communicates the at least one code set value to the EMRS for use in prepopulating the at least one data entry form.

The invention also broadly comprises a method for managing care guidelines in a network, including the steps of: identifying the selection, in a first electronic medical records system (EMRS) in a first location in the network, of a first condition during a visit at the first location and in response to the identifying the first condition, displaying, at the first location, a first treatment plan, from a plurality of treatment plans, regarding the first condition. The steps of identifying and displaying, and the remaining steps of the method, are performed by at least one specially programmed general-purpose computer. In some aspects, the network includes a second location with a second EMRS. Then, the method identifies the selection, in the second EMRS, of the first condition during a first visit at the second location and displays, at the second location, the first treatment plan. In some aspects, the plurality of treatment plans includes a second treatment plan and the method displays, at the second location, the second treatment plan. In some aspects, the plurality of treatment plans includes a third treatment plan. Then, the method identifies the selection, in the second EMRS, of a second condition during a second visit at the second location and displays, at the second location, the third treatment plan.

The invention further broadly comprises a computer-based system for processing work flow regarding a medical visit.

The invention still further broadly comprises a computer-based system for managing care guidelines in a network.

It is a general object of the present invention to provide a method and system for providing a treatment plan to an MP in a single window or display.

It is another object of the present invention to provide a method and system for providing a treatment plan to an MP in response to nominal interaction with an EMRS.

It is still another object of the present invention to provide a method and system for providing a treatment plan to an MP that designates default items.

It is yet another object of the present invention a method and system for providing a treatment plan to an MP that reflects the preferences of the MP and is editable by the MP.

It is a further object of the present invention to provide a method and system for providing respective treatment plans in respective single windows or displays to respective MPs at respective locations in a network.

These and other objects and advantages of the present invention will be readily appreciable from the following description of preferred embodiments of the invention and from the accompanying drawings and claims.

### BRIEF DESCRIPTION OF THE DRAWINGS

FIG. 1 is a block diagram of a present invention system for processing work flow regarding a medical visit and a present invention system for processing work flow regarding a medical visit in a network;

FIG. 2 is a picture of a window generated by an EMRS used with the present invention;

FIGS. 3 through 5 are pictures of windows generated by the present invention;

FIG. 6 is a picture of a window generated by an EMRS used with the present invention;

FIGS. 7 and 8 are pictures of windows generated by the present invention;

FIG. 9 is a picture of a window generated by an EMRS used with the present invention;

FIGS. 10 through 13 are pictures of windows generated by the present invention; and,

FIGS. 14 through 16 are pictures of windows generated by an EMRS used with the present invention.

### DETAILED DESCRIPTION OF THE PREFERRED EMBODIMENT

At the outset, it should be appreciated that like drawing numbers on different drawing views identify identical, or functionally similar elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

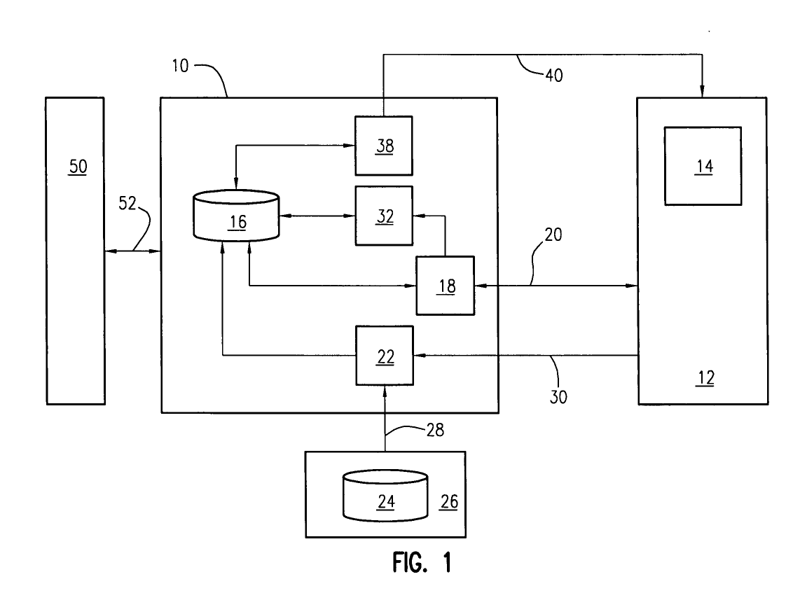

FIG. 1 is a block diagram of present invention system 10 for processing work flow regarding a medical visit and a present invention system for processing work flow regarding a medical visit in a network. In general, system 10 aggregates the multiple actions, screens, data entries, and interactions associated with a medical visit to concisely present information and execute orders. In general, system 10 is associated with or used with an electronic medical records system (EMRS) 12. For example, system 10 can be used with the EMRS incorporated supra. By EMRS, we mean any electronic system used during a medical visit, used to manage workflow in a medical setting, or used to manage medical records. System 10 is not limited to use with any particular EMRS and it should be understood that the use of system 10 with any EMRS known in the art is within the spirit and scope of the invention as claimed. System 10 can be linked to, combined with, or functionally interfaced with EMRS 12 by any means known in the art. For the sake of a simplified presentation, EMRS 12 is shown as a separate element outside system 10, however, it should be understood that other arrangements are possible, for example, system 10 could be shown as nested within EMRS 12. In general and as further described infra, system 10 provides windows to EMRS 12 regarding conditions and treatment plans. EMRS 12 then displays the windows, typically on a graphical user interface (GUI), such as GUI 14.

System 10 includes library 16, which stores, maintains and operates upon a plurality of treatment plans relating to respective medical conditions or problems. Each treatment plan is composed of guidelines and items associated with the respective problem or complaint. Hereinafter, the terms guidelines and items are used interchangeably. The items stored by library 16 include, but are not limited to, the following:
History Documenters: data entry form(s) used to document the history of a problem and the most common answers to the questions contained within them for patients with this problem.
Exam Findings Profiles: show which body areas are physically examined when seeing a patient with this problem, and, what are the most common answers to the questions contained within the related data entry forms for patients with this problem.
Procedures: procedures performed for this problem.
Labs: lab tests ordered when a patient is seen for this problem.
Imaging Tests: radiology requisitions ordered when seeing this problem.
Handouts: handouts given to a patient with this problem.
Letters/Notes: notes given to a patient with this problem.
Instructions: verbal instructions given to the patient for this problem.
Medications: prescriptions written for a patient with this problem.
Referrals: to whom (what type of specialist(s)) patients with this problem are referred.
Alerts: information presented to the doctor as a reminder when seeing a patient with this problem.

System 10 includes interface element 18, used to link system 10 to EMRS 12. In FIG. 1, most interaction between system 10 and EMRS 12 passes through element 18, however, it should be understood that other arrangements are included within the spirit and scope of the invention as claimed. For example, functional blocks in system 10, further described infra, can be shown as directly linked to EMRS 12. To launch system 10, element 18 identifies on line 20, the selection, in EMRS 12, of a condition. Typically, this selection is made by an MP during a patient visit. The selection can be made by the MP with respect to treating the patient, documenting the visit, or billing for the visit. However, it should be understood that the selection can be with respect to other aspects of the EMRS and the identification of such selections is included in the spirit and scope of the claimed invention. Thus, system 10 is launched within the existing framework of the EMRS. That is, there are no special commands required and no extra effort is required of the MP to launch system 10.

In some aspects, GUI 14 is accessed to identify the selection. In response to the identification on line 20, the library selects or generates a treatment plan from the plurality of treatment plans and items in storage. Library 16 then provides the selected treatment plan to element 18, which provides the selected element to EMRS 12 on line 20 for display, for example, on GUI 14.

In some aspects, system 10 also includes populating element 22, which in some aspects, is used to populate library 16. Sources for information in library 16 include, but are not limited to, established standards. That is, such standards are used to at least partially populate the library. Various standards have been created by national, regional, and local organizations establishing recommended treatments for particular types of diseases or medical conditions. For example, the American Diabetes Association generates guidelines for the treatment of diabetics. Also, local physician associations, insurers, etc. create care guidelines for various diseases, such as Asthma, hypertension, etc. An established standard is shown in storage element 24 for organization 26, located external to system 10. In the case of established standards, the data would ordinarily be imported, for example, on line 28, and therefore supplied to the practice, as shown in FIG. 1. The data could, however, be manually entered. It should be understood that other arrangements of storage elements and organizations storing and supplying standards of treatment are included in the spirit and scope of the invention as claimed. For example, multiple storage elements are possible for a single organization and system 10 can be linked with multiple organizations.

In some aspects, element 22 accepts data from the EMRS on line 30 and uses this data to at least partially populate library 16.

System 10 includes editing element 32. MPs can customize library 16 to reflect their unique preferences and routine in regard to treatment and documentation through element 32. For example, in some aspects, and as shown infra, library 16 is modified through the interaction of system 10 with MPs through input supplied by the MPs through EMRS 12. For example, editing element 32 can accept an editing input via line 20 and library 16 modifies a particular treatment plan in response to the editing input.

In some aspects, library 16 designates a specified item or guideline in library 16 as a default guideline. Default guidelines are further discussed infra. In some aspects, an item is designated as a default item in response to an input to system 10 or in accord with a standard of treatment used to populate library 16. In the first case, library 16 accepts an input from a user via line 20 and marks the specified item in response to the input. In the second case, the standard of treatment prioritizes possible items and provides the criteria for determining if an item should be designated as default. In some aspects, library 16 saves the default designation with respect to the specified item. For example, the next time that library 16 displays a treatment plan including the specified item, the specified item is presented as a default item.

In some aspects, system 10 includes execution element 38. Element 38 executes items from the subject treatment plan. That is, element 38 carries out tasks associated with the treatment plan. Typically, element 38 executes the items when a user exits system 10. However, it should be understood that other arrangements are possible, for example, element 38 could execute items as these items are displayed or selected by a user of system 10. In general, element 38 accesses systems outside system 10 to execute the subject items, for example, line 40 to EMRS 12. Items that can be executed by element 38 include, but are not limited to: sending information to other parties; generating medication prescriptions; creating letters regarding the patient; providing written materials for the patient; generating medical service orders; triggering procedures to be performed; and generating a history and documenting examination findings

In some aspects, EMRS 12 includes a plurality of data entry forms, for example, Forms 14 through 16. In response to element 18 identifying the condition, library 16 generates at least one code set value regarding the condition, element 18 sends the at least one code set value to EMRS 12, and the at least one code set value is available to EMRS 12 to prepopulate the appropriate data entry forms. That is, the values are used to select/fill-in applicable portions of the forms. This process is further described infra.

Because system 10 maintains library 16 including the information described supra, all the relevant and necessary aspects regarding a patient visit are presented to the doctor immediately, in a single window or form, as soon as a problem or complaint is identified. Specifically, system 10 reminds the MP of the recommended treatment plan for the selected problem and relieves the MP of the necessity of selecting, opening, and interacting with a myriad of windows in order to execute the multiple tasks associated with a medical visit.

It should be understood that any means known in the art can be used to link library 16 with other elements, components, and systems. For example, hard wire, coaxial, wireless, or radio frequency links can be used. It also should be understood that the components in system 10 represent functions of the system and that other arrangements of functional elements are possible. For example, the functions of element 18 can be incorporated in library 16 and element 18 removed from a representation such as FIG. 1. In general, it should be understood that any means known in the art can be used to implement and represent the functionality of the claimed invention.

The present invention also is applicable to network applications. In a network application, system 10 is connected to a plurality of locations in a network. For example, EMRS 50 represents a second location in a network connected to system 10. In some aspects, different locations in the network are separately disposed. That is, the locations are located in different locations or are physically separate one from the other. In some aspects, EMRS 12 and 50 are the same, that is, in both locations the same EMRS is used. In some aspects, EMRS 12 and 54 are different. The present invention is not limited to any particular number, combination, or ratio of same or different EMRSs. In general, the discussion supra regarding system 10 and EMRS 12 is applicable to a network application. EMRS 50, in general, is similar to EMRS 12, with respect to function with system 10, although it should be understood that other variations or differences are otherwise possible for locations within a network application. Thus, in a network application, system 10 services a plurality of locations and computer systems and can be considered a central library. EMRS 50 is linked to library 16 via line 52. Line 52 includes some or all of the functionality of the lines connecting EMRS 12 and system 10 and for the sake of simplicity are not fully detailed. In a network application, system 10 is linked to any number of locations and identifies and responds to the selection of multiple conditions at multiple sites.

One advantage of a network application is the control of treatment plans among a plurality of locations and respective computer systems. For example, controlling the treatment plan selected and displayed for a specific medical condition among the locations. Thus, in some aspects, at any computer system within the network, a condition input is accepted, and a centralized, specified treatment plan is presented to all computer systems within the network, for example, the same treatment plan is displayed at EMRS 12 and 50 for a same condition input to the library. Alternatively, specific treatment plans can be selected for various groupings of computer systems within the network. For example, in response to the same medical condition, a first treatment plan can be selected from the library and displayed for computer systems in a first geographic area and a second treatment plan can be selected from the library and displayed for computer systems in a second geographic area. Thus, different treatment plans can be displayed at EMRS 12 and 50 for a same condition input to the library.

In general, the discussion regarding default guidelines for system 10 is applicable to a network application. However, it should be understood, that just as system 10 can select different treatment plans for a same problem in a network application, system 10 can designate, as default guidelines, different guidelines in a same treatment plan, depending on the computer system receiving the treatment plan. For example, EMRS 12 and 50 could receive different treatment plans for the same condition. In general, the discussion regarding execution of guidelines in system 10 is applicable to a network application. However, it should be understood that the functions of executing element 38 can be incorporated in whole or in part in one or more of the locations in a network.

A network application of the present invention can be used to distribute community standards for treatment among a wide variety and number of medical offices and institutions. A network application also can be used to distribute care guidelines to various medical offices and EMRSs. Organizations controlling payment to MPs, for example, insurance companies, can use system 10 to ensure the uniform and appropriate treatment plans are provided, across a network, for MPs treating the same medical conditions or problems.

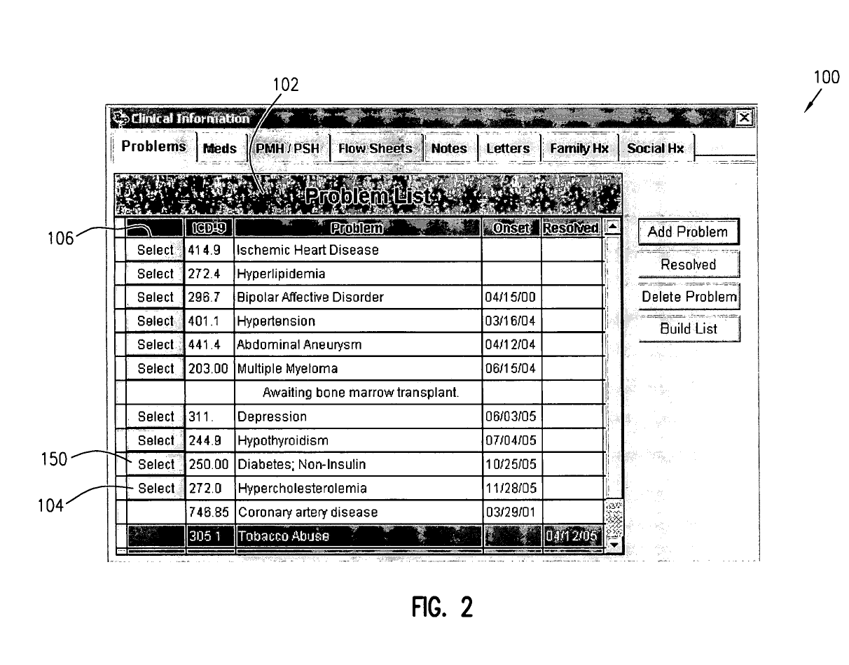

FIG. 2 is a picture of a window generated by an EMRS used with the present invention. The following discussion is with respect to an example for a fictitious patient X. Window 100 displays problem list 102 generated by the EMRS. In some aspects, an MP accesses system 10 via such a problem list. In the example of patient X, since this is a follow-up visit, the problem for which they are being seen would have previously been added to Problem List 102. In FIG. 2, the MP has selected button 104 regarding Hypercholesterolemia, the problem identified regarding the patient. In FIG. 2, selecting one of the buttons in row 106 launches system 10. It should be understood that system 10 can be launched using any means known in the art and that such means are within the spirit and scope of the invention as claimed.

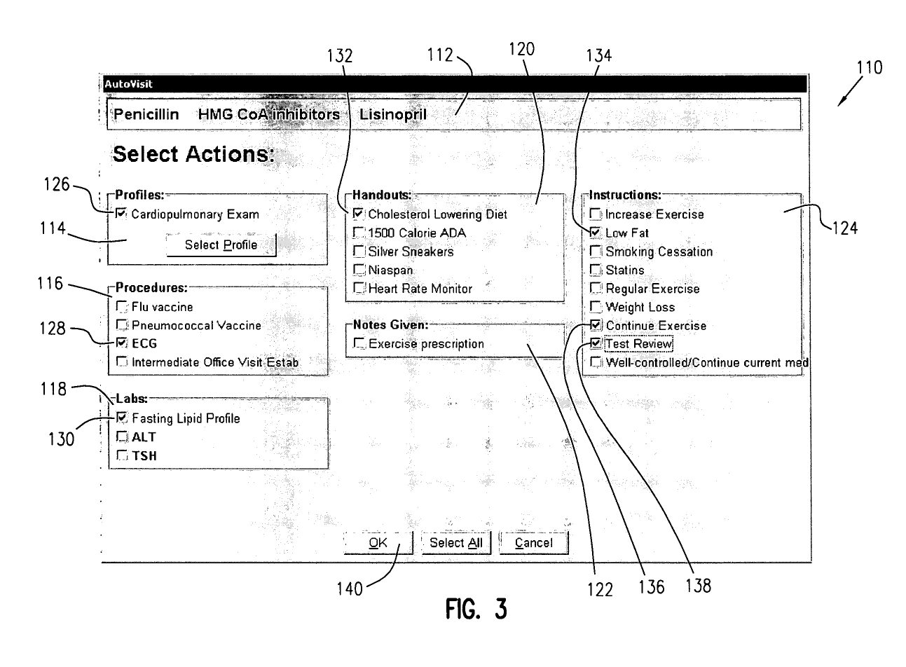

FIGS. 3 through 5 are pictures windows generated by the present invention. The following should be viewed in light of FIGS. 1 through 5. Window 110 in FIG. 3 was generated by clicking button 104 in the previous screen. Patient X's drug allergies are listed in box 112. The remaining guidelines or visit items are shown below box 112. Screen 110 includes profile 114, procedures 116, labs 118, handouts 120, notes given 122, and instructions 124. However, it should understood that the number and type of items shown and the size, content, and appearance of window 110 can vary depending on the problem selected in window 100 and that other configurations of window 110 are included in the spirit and scope of the invention as claimed. Each of the items has a check box next to it. More than suggestions, these items are actionable. This enables the MP to choose which of the routinely performed items related to the problem selected in window 100 should apply to the example visit. That is, the MP is presented with a list of selected components for a visit of this type and the MP determines which ones of the items are appropriate for the patient being seen.

Certain of the items in window 110 are already marked when window 110 is displayed. These are referred to as default guidelines or items. As explained infra, the default items are marked or selected based upon guidelines in a library of treatment plans or previous actions taken by an MP addressing the medical condition in question (in this example, Hypercholesterolemia). In window 110, some of boxes 126, 128, 130, 132, 134, 136, and 138 are default items.

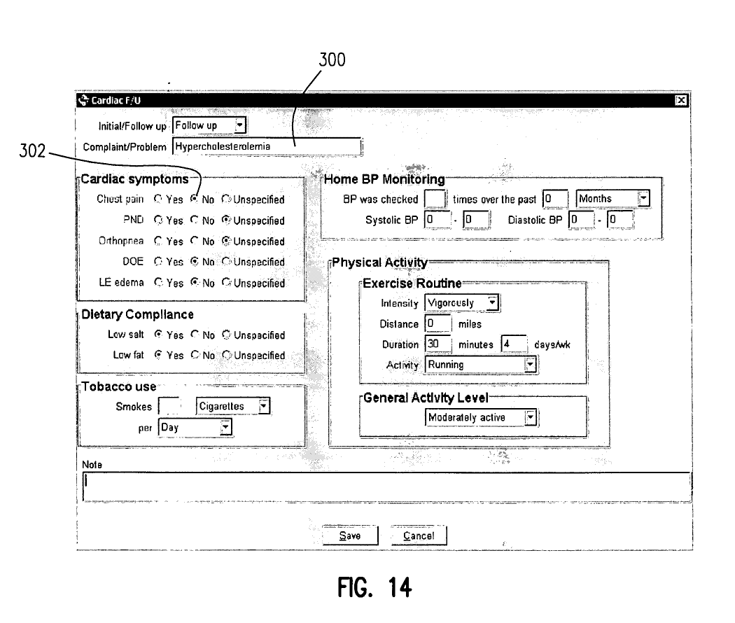

The MP exits window 110 by selecting button 140. After closing window 110, system 10 automatically opens pages or forms equivalent to the pages and forms discussed in the Background Section. For example, a Cardiac Follow-Up history form, as shown in FIG. 14, is triggered to open immediately after the window 110 is closed. The history form is part of the EMRS. The history form, in this case, the Cardiac Follow-Up history form, facilitates entry of the patient's history in regards to Hypercholesterolemia and adds it to the subjective part of the note. In some aspects, the link to the history documenter is automatic; it is not a selectable option in window 110. In general, some type of history form related to the condition in question is generated when the MP exits system 10.

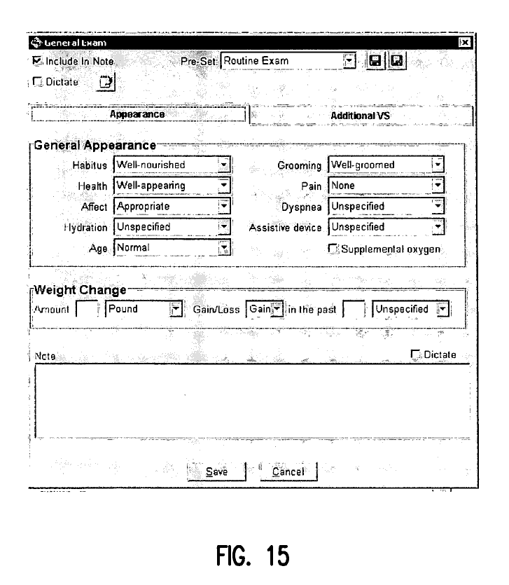

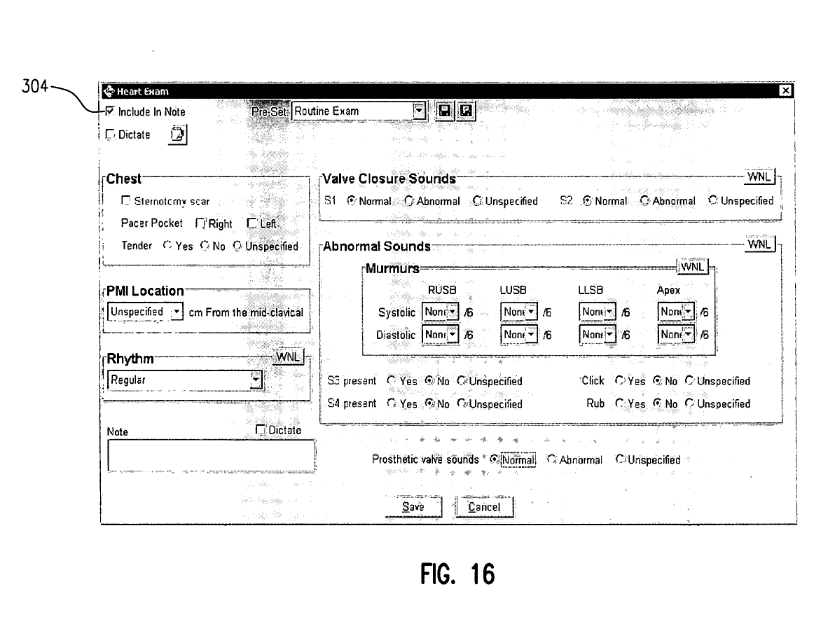

For the present example, Cardiopulmonary Exam Profile 126 is selected. As a result, when button 140 is selected, EMRS forms for General, Neck, Heart, Chest, and Extremities are selected and “typical” or “expected” results are entered in response to the questions contained in those forms. For example, FIGS. 15 and 16 show General Exam and Heart Exam forms, respectively. Thus, the entire objective part of the note being completed without any data entry required of the doctor. It is only necessary to open one of the exam findings forms associated with an EMRS when there is an exception—an atypical finding—to be documented. For example, if the MP detected an unexpected result during the Neck exam, the MP can access the Neck examination form in the EMRS and change the value or entry pertaining to the unexpected result.

Because box 128 is checked, an EKG is automatically ordered. As a result, a message is sent to another MP to perform an EKG on this patient; the billing system is notified to bill for an EKG as part of this patient's claim; and the fact that an EKG was performed is noted in the Assessment and Plan section of note for the current visit.

Because box 118 is checked, a Fasting Lipid Profile lab is ordered. As a result, a lab requisition is printed automatically; the patient's medical record is updated noting this order; the patient's preventive care/disease management record is updated; and notation is made in the Assessment and Plan section of the note for the current visit.

Because box 132 is checked, a Cholesterol Lowering Diet handout is automatically printed for the patient. The handout also is documented in the Assessment and Plan section of the note for the current visit.

The note for the current visit also will document the fact that the MP recommended the patient to be on a low fat diet, continue his current exercise routine, and the fact that the doctor reviewed with the patient the results of the last tests he had taken.

Essentially, everything the MP needs to order and document is done for her with just a few clicks within one window, for example, window 110. Where there are atypical findings, the MP can open up the appropriate windows to complement the documentation completed by system 10. However, it is possible that the doctor needs to do nothing extra as pertains to orders and documentation. The actions described supra were triggered by the MP clicking on a single box in window 100.

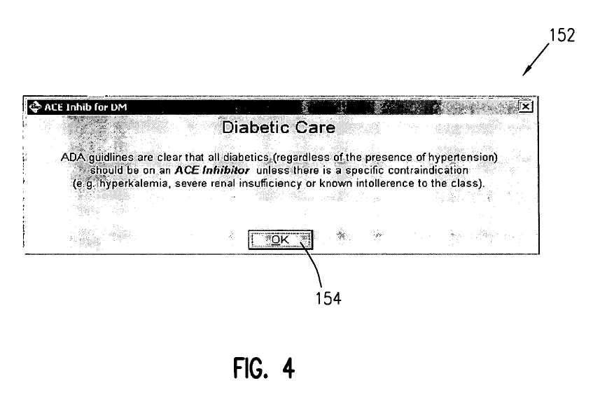

To cause FIG. 4 to be generated, the MP returns to window 100 and selects button 150 for Diabetes. Often, a patient is seen for multiple problems. In the present example, patient X also a diabetic and is being seen for follow-up of Diabetes. Again, system 10 is engaged. However, in this example, system 10 automatically displays Alert window 152 as shown in FIG. 4.

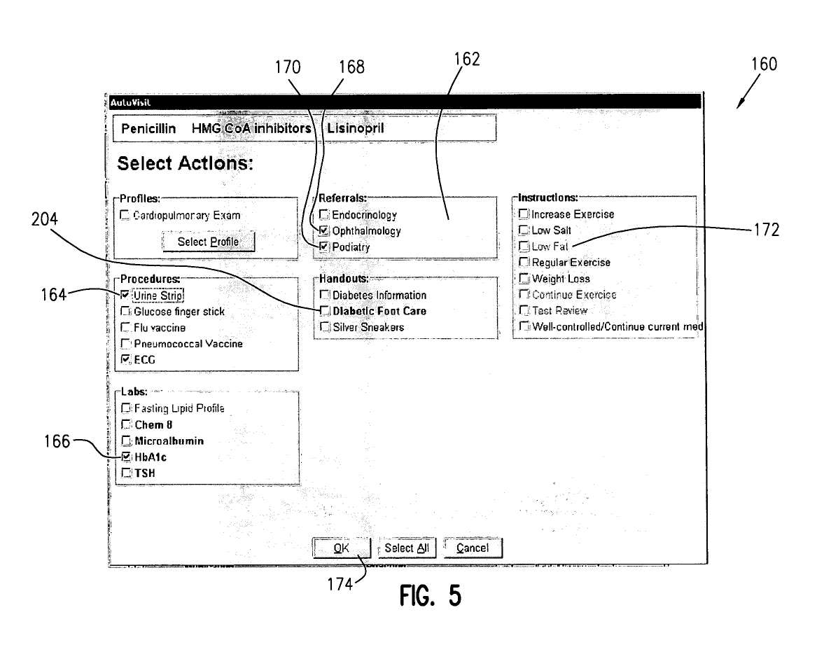

The MP clicks on button 154 in window 152 to reveal window 160 in FIG. 5. Window 160 is slightly different than window 110, since window 160 is addressing a different medical condition than that addressed by window 110. Specifically, window 160 contains additional procedures, labs and handouts, but most notably, it contains a section for Referrals 162. A referral box was not included in window 110, since such a box was not appropriate for the condition addressed in window 110. The selected guidelines in window 160 are Urine Strip 164, a procedure done in the office, HbA1c lab test 166, Ophthalmologist and Podiatrist referrals 168 and 170. Some of these guidelines are default guidelines and some are selected by the MP.

In some aspects, items appearing in window for system are color-coded. Items in a first color, for example, green, are items recommended for treating the condition addressed by a current window, but previously selected for this patient for another problem. For example, Low Fat 172 is shown in the first color. Items in a second color, for example, blue, are tests or procedures that have been previously ordered but for which results have not been reported back to the MP's office. In the case of the labs, the second colors serves as a reminder to the MP that he has not received results. This enables the MP to determine if such lack of results indicates other problems, such as a failure of the patient to have the test or procedure performed. Items in a third color, for example, red, are overdue items from the patient's preventive care/disease management profile. Different colors can apply to the same item and rules are established in system 10 regarding which color to display in such cases.

The MP exits window 160 by selecting button 174. Because window 160 includes referrals to specialists, upon closing window 160, the MP is presented with a window (not shown) displaying lists of Ophthalmologists and Podiatrists from which to choose to order an eye exam and foot care, respectively. Selecting these referrals has the following results: referral slips are printed, giving information to the patient such as the name, address, and phone number of the specialists to whom the patient is referred; the patient's medical record is updated; the billing system is notified of a pending referral which may notify the insurance company for authorization; a complete medical history of the patient is prepared by the system to be sent automatically to the specialists; and the referrals are noted in the Assessment and Plan section of note for the current visit.

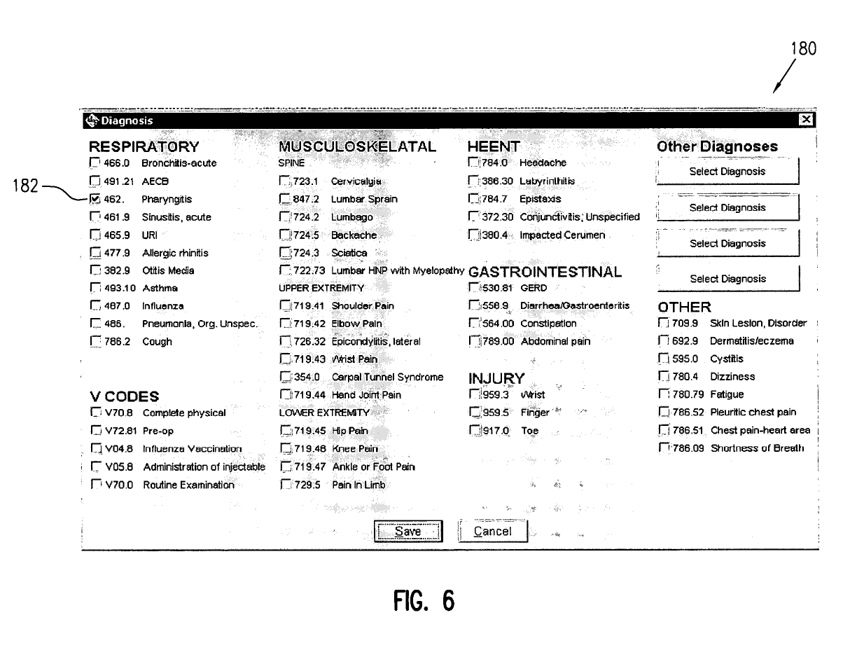

FIG. 6 is a picture of a window generated by an EMRS used with the present invention. The following should be viewed in light of FIGS. 1 through 6. To cause FIG. 6 to be generated, the MP returns to the EMRS. Window 180 in FIG. 6 is a typical diagnosis form in an EMRS. System 10 also addresses new conditions, that is, conditions for which a patient has not been seen in the past and conditions regarding which the patient has no record in system 10. For example, patient X complains of a sore throat. To address this new problem, in some aspects, the MP returns to the EMRS to a diagnosis form, such as window 180 in FIG. 6 and selects Pharyngitis button 182. Such windows contains a list of common acute problems for which a particular practice may see patients. It should be understood that other means known in the art can be used by system 10 to initiate a response to a new condition and that the use of such means is within the spirit and scope of the invention as claimed.

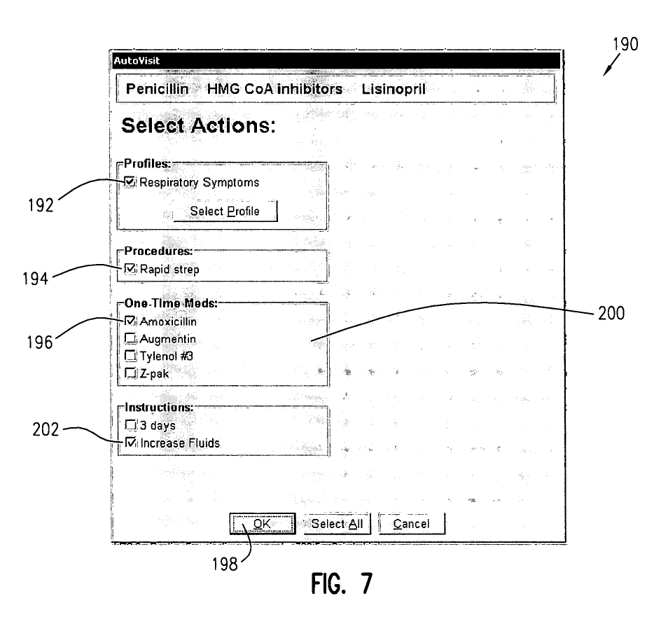

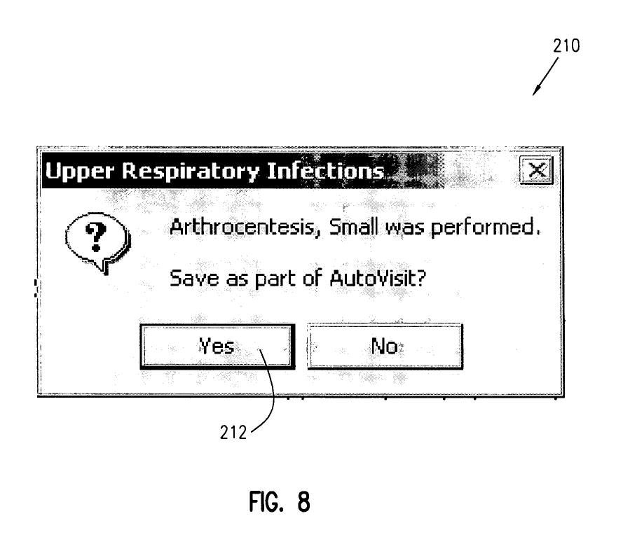

FIGS. 7 and 8 are pictures of windows generated by the present invention. The following should be viewed in light of FIGS. 1 through 8. The MP selected checkbox 182 to generate window 190 in FIG. 7. That is, selecting box 182 launches system 10 with Pharyngitis as the condition to be addressed. Window 190 also is displayed if the MP adds Pharyngitis to window 100 and selects an associated button in column 106. In window 190, the following items are marked: Respiratory Symptoms Profile 192, Rapid Strep 194, and Amoxicillin 196. The MP exits window 190 by selecting button 198. After closing window 190, system 10 automatically accesses, for Profile 192, pages or forms equivalent to the pages and forms discussed in the Background Section. For example, areas to be examined in this profile include General, Chest, and HEENT (Head, Ears, Eyes, Nose and Throat). As in the example of FIG. 3, the most common findings of these examinations for a patient with the subject condition, in this case a sore throat, are automatically documented in the related windows. System 10 orders Rapid Strep 194, which can be an in-house procedure. In such cases, system 10 an electronic message to the MP performing this procedure (e.g., “Patient X in room number 5 needs a Rapid Strep”). Additionally, the billing system is notified so the patient's insurance can be billed for the procedure, and, the fact that it was done is documented on the note. Amoxicillin 196 is prescribed. For this particular condition, section 200 for “One-Time Meds” appears, since such medication guidelines are part of the library for system 10. System 10 causes a prescription to be printed (or sent electronically if the pharmacy has that capability), adds Amoxicillin to the patient's medications history, and documents it in the note. In addition, box 202 advises the MP to recommend increased fluid intake for the patient. The note will indicate that the MP did so.

Patient X was seen for three problems: follow-up for Hypercholesterolemia and Diabetes, both of which were selected by the MP from Problem List 102, and Pharyngitis, an acute problem which the MP selected from her EMRS. In each case, once the problem or conditions was selected, system 10 was launched and displayed a recommended course of action regarding the patient's treatment and provided documentation of the note. As note supra, certain of the items or guidelines in windows 110, 160, and 190 were checked by default. That is, these items are “pre-selected.” In some aspects, a default item is strongly recommended or routinely performed. The remaining, unchecked items may be applicable, but not routinely performed. The MP has the option of accepting the default items, unselecting the default items, or selecting other “non-default” items in a particular window. Once the MP is satisfied with the selected items, he exits the window and system 10 executes the guidelines or items selected.

Thus, for each of the three conditions, the doctor selected the items he felt pertinent for this patient by checking the boxes, then clicked the respective OK button. As a result:
The MP was given an alert in regards to treating Diabetes.
The MP was reminded of all of the recommended options for treating the three problems.
The MP was able to quickly document the history of the problems.
The relevant areas (General, Heart, Neck, Extremities, Head, Ears, Eyes, Nose, and Throat) were flagged as examined with findings documented that were typical of the three problems for which the patient presented.
The following procedures were documented as being performed, billed, and appropriate notification given to the nurse: EKG, Urine Strip, and Rapid Strep.
Lab requisitions were printed for Fasting Lipid Profile and HbA1c.
Two handouts were printed for the patient.
A prescription for Amoxicillin was printed.
Referrals to an Ophthalmologist and podiatrist were initiated (with complete medical histories sent to the specialists) and information slips given to the patient.
The patient's medical record was automatically updated in regards to the orders (labs, prescription, referrals, and procedures).
The patient's Preventive Care/Disease Management profile was updated for all of the above where appropriate.
A note was automatically generated.

Often times the MP does not know to what extent he will follow the recommendations/options presented to him in a particular system 10 window. For example, some of the items may be determined to be appropriate only after further examination of the patient. For this reason, not only does system 10 launch an appropriate window when a problem or condition is first selected, for example, as shown in FIG. 2, but system 10 also can be re-opened at anytime during a visit to view and select other options.

As noted supra, system 10 includes library 16 of treatment plans. As noted in the description for FIG. 1, library 16 can be modified by interaction between an MP and system 10. That is, system 10 can be trained. Specifically, system 10 can be trained to add a guideline or item to library 16 or to designate an existing guideline or item in library 16 as a default item. In some aspects, certain of the MPs using system 10 are designated as trainers and system 10 uses input from the trainers to accomplish the training described above. As described supra, when system 10 opens a window, certain of the items in the window, that is, default items, may be “pre-selected” by system 10. Also as described supra, an MP can de-select default items or can select items that are not pre-selected. For a trainer, system 10 is configured to identify changes regarding selected items and to query the trainer as to whether the changes should be incorporated in the library and to make changes to the library responsive to the trainer's input with respect to the query.

System 10 generates window 210 in FIG. 8 in response to actions taken by a trainer. The following describes the addition of an item to library 16. For example, as part of the treatment of an Upper Respiratory Infection for a patient, the MP determines that an Arthrocentesis is necessary. In this case, an Arthrocentesis is not included in library 16, that is, an Arthrocentesis is not listed in window 190 in FIG. 7. Therefore, the MP returns to the EMRS to access a procedure window (not shown) listing Arthrocentesis and selects Arthrocentesis. Upon exiting the window regarding the Arthrocentesis, system 10 displays window 210. In window 210, system 10 provides the MP the opportunity to add the “new” item to library 16. If the MP selects Yes 212, the guideline or procedure, in this case, Arthrocentesis, and information regarding the guideline or procedure is added to library 16 regarding the treatment of Upper Respiratory Infections. In general, this information is provided by the EMRS. In this manner, system 10 learns the habits of the individual MP, how they treat specific problems, and remembers selected guidelines or items for future use. Thus, any time an item is selected and that item is not already in the library, the system gives the MP the opportunity to update the library (to add the action to the library) with a single click. This item can be a procedure, a medication, a referral, a lab, an imaging study, a handout, a verbal instruction given, a note printed, or any other item included in the EMRS. Thus, over a short period of time, library 16 becomes well populated with the doctor's preferences and routine.

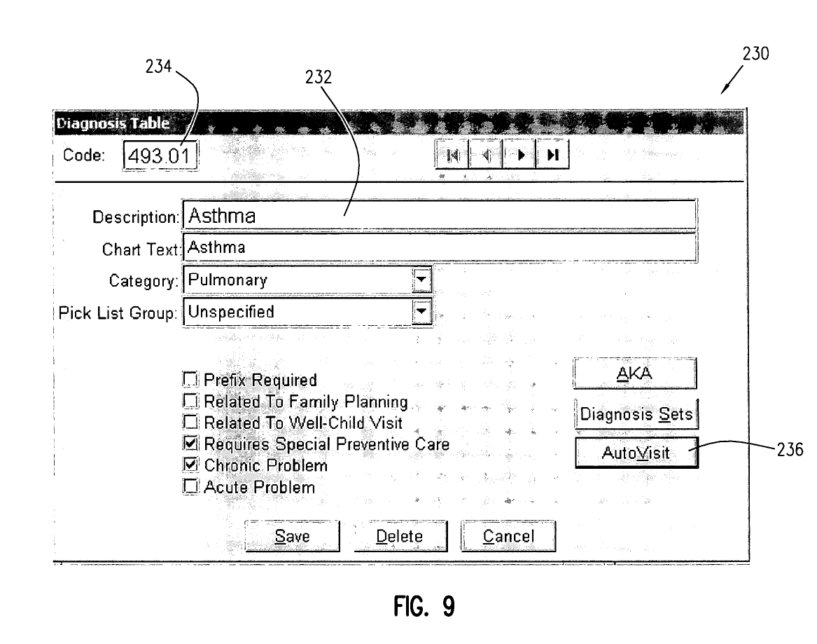

FIG. 9 is a picture of a window generated by an EMRS used with the present invention. The following should be viewed in light of FIGS. 1 through 9. Window 230 in FIG. 9 shows a diagnosis table for an EMRS with which system 10 is being used. In some aspects, library 16 is a table of items linked to respective diagnosis codes, for example ICD-9 codes. In some aspects, library 16 is accessed from an EMRS using medical record forms or table maintenance forms for the EMRS. In either case, it is first required that a diagnosis be selected. Then, a user can access system 10 to view and update library 16 regarding the diagnosis code in question. “Asthma” was selected for box 232. The ICD-9 code for Asthma is 493.01 and is shown in text box 234. To access library 16 regarding code 493.01, button 236 is selected.

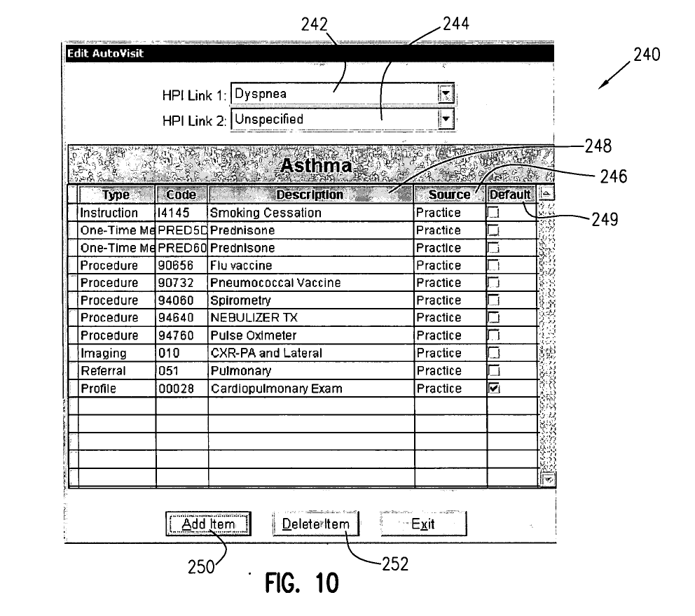

FIGS. 10 through 13 are pictures of windows generated by the present invention. The following should be viewed in light of FIGS. 1 through 13. Window 240 in FIG. 10 is generated by selecting button 236 in the preceding figure. Window 240 displays the treatment plan in library 16 for Asthma. History of Present Illness (HPI) Links 242 and 244 at the top of the window allow the user to select the appropriate form documenter(s) to record the patient's history in regards to this problem. For example, for window 110 in FIG. 3, the HPI is a Cardiac Follow-Up history form (FIG. 14). Source column 246 indicates the origin of each guideline or item in column 248. In window 240, all the items in column 248 are noted as “practice.” That is, the items were added as practice preferences, probably as a result of system 10's self-learning capability. For items originating from a standard of treatment, the organization generating the standard would typically be indicated. For example, an item originating from the guidelines of the American Diabetes Association could be shown as “ADA” in column 246. Checkboxes in Default column 249 indicate which of the items will be checked by default when the doctor is presented with the system 10 window, in this case, when a patient is being seen for Asthma. Default boxes or items are further described supra. Items are added to or deleted from column 248 by selecting buttons 250 and 252, respectively.

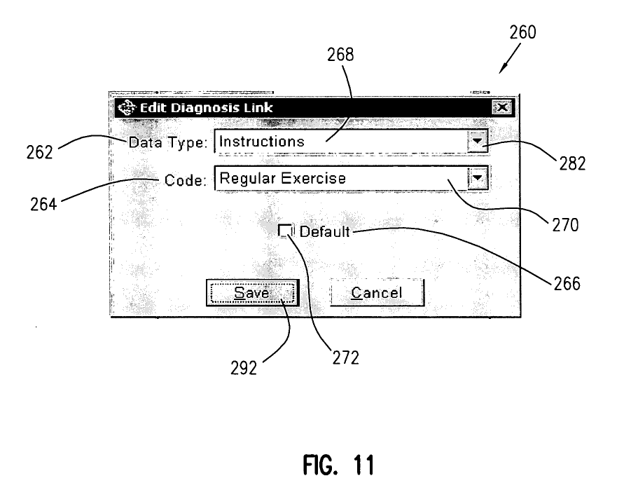

Window 260 in FIG. 11 was generated by selecting button 250 in window 240. Window 260 has three components Data Type 262, Code 264, and Default 266. Data type describes the new item. For example, in FIG. 11, the item is an instruction. Some, but not all, possible entries for drop-down box 268 are shown in the present figure. The entries in box 268 determine the contents of Code drop-down box 270. Code 264 sets the specific element of the family determined by the data type. That is, once an entry is selected for box 268, box 270 is automatically populated with the appropriate entries from library 16. Box 272 indicates whether the specified item is designated as a default item when system 10 displays a treatment plan including the specified item

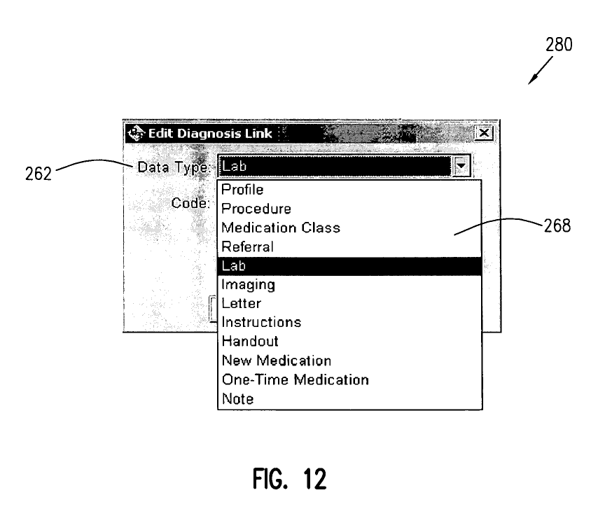

Window 280 in FIG. 12 was generated by selecting button 282 in FIG. 11. Some, but not all, possible entries for data type 262 are shown in box 268. The data types shown in window 268 are as follows:

Procedure—indicates a type of procedure is performed. In real-time use, selecting one: updates the billing system that the procedure was performed, sends an electronic message to the nurse (if it is a procedure she performs), updates the patient's Preventive Care or Disease Management profile (where applicable), and documents it in the note.

Medication Class—in real-time use, selecting one causes a window to appear from which the doctor can select a medication of this class to prescribe to the patient. This causes: a prescription to be printed (or sent electronically to the pharmacy), the patient's Medications List is updated, and the medication is documented in the visit note.

Referral—in real-time use this causes a window to appear of specialists of this type from which the doctor can select to refer the patient. This results in: the patient's Referral List is updated, a Referral Slip is printed (with the specialist's name, address, and phone number), a summary of the patient's medical record is prepared for transmittal to the specialist, and the referral is indicated in the note.

Lab—indicates a specific lab is to be ordered. In real-time use: the patient's Lab Order List is updated, a Lab Requisition is printed (or transmitted electronically), the patient's Preventive Care or Disease Management profile is updated (where applicable), and the order is indicated in the note.

Imaging—indicates a specific radiology study is to be ordered. In real-time use, the patient's Radiology Order List is updated, a Radiology Requisition is printed, the patient's Preventive Care or Disease Management profile is updated (where applicable), and the order is indicated in the note.

Letter—indicates a specific note for the patient is to be printed (e.g., Work Excuse). In real-time use, the letter is automatically printed and the fact that it was given to the patient is indicated in the note.

Instruction—documents that the patient was given specific verbal instructions. In real-time use the full text of the instructions are documented in the note as having been given to the patient.

Handout—causes a Handout to be printed to be given to the patient and the fact that it was given is indicated in the note.

New Medication—indicates a specific medication that is to be prescribed. In real-time use this causes: a prescription to be printed (or sent electronically to the pharmacy), the patient's Medications List is updated, and the medication is documented in the visit note.

One-Time Medication—indicates a specific one-time med is to be prescribed. This has the same results of a new medication above.

Profiles—Profiles are powerful documenter tools. They cause various areas of examination (e.g., heart, lungs, skin, etc.) to be indicated as examined. Each area has a host of specific data elements (e.g., the heart's rhythm), each with its own regular values. In real-time use, a profile allows for an immediate indication made of what the doctor examined and what the detailed findings were of each area. From there the doctor can quickly make changes to atypical findings. The EMRS's text generation engine compiles all of the data into text for the visit note. They thus link the typical types of examination, all with their own regular values, to the diagnosis.

Note—an alert text box that contains up-to-date information the doctor should be familiar with when he selects that diagnosis.

HPI Links—indicate the nature of the problem and how it is to be documented. In real-time use this causes the correct History of Present Illness window to appear to expedite the entry of this problem's history (how, when it happened, its severity, frequency, etc.).

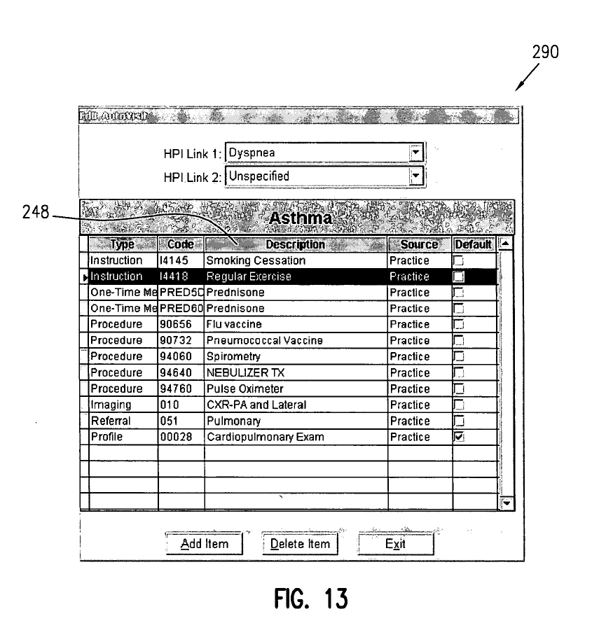

Window 290 in FIG. 13 was generated by selecting button 292 in FIG. 11. Selecting the data type determines the contents of the Code selection in menu 270. For example, if Lab is selected for box 268, box 270 for Code is populated with all the labs on file from which the MP can select the specific lab or labs desired. In FIG. 11, Instructions and then Regular Exercise were selected. In response to clicking Save 292, system 10 generates window 290. Regular Exercise has been added to column 248. That is, Regular Exercise has been added to the display shown in FIG. 10. In some aspects, whether data is manually added to a table, as in the above example, data is collected through system 10's self-learning process, or data is imported from an outside source, the data appears in library 16 in the format shown supra.

FIGS. 2 through 13 are an example regarding a follow-up visit for a fictitious male patient X. Although FIGS. 2 through 13 are regarding specific medical conditions, it should be understood that system 10 is not limited to any particular medical condition or combination of medical conditions and that the use of system 10 with any medical conditions known in the art is within the spirit and scope of the invention as claimed. It also should be understood that system 10 is not limited for use with respect to any particular type of patients.

FIGS. 14 through 16 are pictures of windows generated by an EMRS used with the present invention. As noted supra, system 10 can be used to provide code set values to an EMRS for use in prepopulating data entry forms. As an example, after the use of system 10 in the example of FIGS. 2 through 13, the data entry forms shown in FIGS. 14 through 16 are prepopulated as shown, without any effort from and without requiring any further input from the MP. For example, the text shown in the text boxes, for example text box 300, is supplied, appropriate “Yes” and “No” boxes, for example, box 302, are selected, and appropriate text boxes, for example, box 304, are checked.

The following is a discussion of the technical specification for system 10 and should be viewed in light of FIGS. 1 through 16. As shown in Table 1, library 16 is based (keyed) primarily on either a specific Diagnosis Code or a code representing a Family of Diagnosis Codes. The above represents the essential elements (as it relates to the present invention) of the Diagnosis Table. The Primary Key for the table, the Diagnosis Code itself, is commonly an ICD-9 code, the International Classification of Diseases code set established by the National Center for Health Statistics.

TABLE 1
Diagnosis Table

| | | |
|---|---|---|
| Code | C6 | Primary Key (usually an ICD-9 code) |
| Description | C60 | Name of Diagnosis |

Alternately, the library may be keyed on a family of Diagnosis Codes. Tables 2 And 3 show this approach. Table 2 is a simple list of families, each with a unique code. Table 2 has a one-to-many relationship with Table 3. Table 3 contains a list of every diagnosis code contained with a family. Tables 2 and 3 provide the primary linking mechanism in the library.

TABLE 2
Diagnosis Family Table

| | | |
|---|---|---|
| Code | C5 | Primary Key |
| Description | C40 | Name of Family |

TABLE 3
Diagnosis Family List

| | | |
|---|---|---|
| FamilyCode | C5 | Links to Diagnosis Family Code |
| DiagnosisCode | C6 | Links to Diagnosis Code |

The table layout for library 16 is shown in Table 4. The value of the CodeType field in Table 4 is “1” to indicate the Code is a Diagnosis Code and thus is linked directly to the Diagnosis Table, or “2” to indicate the Code is a Diagnosis Family Code, and thus applies to any Diagnosis Code that is in the Family's List. The ItemType field in Table 4 indicates whether the specific item referred to in ItemCode is a procedure (ItemType value=“PRO”), a lab (ItemType value=“LAB”), an imaging or radiology order (ItemType value=“RAD”), a handout (ItemType value=“HO”), etc. The field identifies what type of item the current record is, and therefore, what reference table to which it is linked.

TABLE 4
Present Invention Library

| | | |
|---|---|---|
| Code | C6 | Links to Diagnosis Family Code or |
| | | Diagnosis Code |
| CodeType | C1 | Indicates whether Code is a single |
| | | diagnosis or a family |
| ItemType | C3 | Indicates what table ItemCode links |
| | | to |
| ItemCode | C11 | Specific item code - links to one of |
| | | several tables (e.g., Procedure, Lab, |
| | | Handout, etc.) |

The layout of the basic order tables are as follows. Table 5, The Procedure Table, contains a list of all in-house procedures that may be performed in a medical practice. Most commonly, the Procedure Code is a CPT code, from the code set of Current Procedural Terminology compiled by the American Medical Association. Additional fields can be present in this table to indicate billing fees, configuration, etc. However, these fields are not particularly relevant to system 10.

TABLE 5
Procedure Table

| | | |
|---|---|---|
| Code | C5 | Primary Key (usually a CPT code) |
| Description | C60 | Name of Procedure |

Table 6 contains a list of lab orders.

TABLE 6
Lab Table

| | | |
|---|---|---|
| Code | C5 | Primary Key |
| Description | C60 | Name of Lab Order |

Table 7 contains a list of imaging or radiology orders.

TABLE 7
Imaging Table

| | | |
|---|---|---|
| Code | C5 | Primary Key |
| Description | C60 | Name of Imaging/Radiology Order |

Table 8 contains a list of handouts the practice uses. Most are linked to a document that would get printed on demand.

TABLE 8
Handout Table

| | | |
|---|---|---|
| Code | C5 | Primary Key |
| Description | C60 | Name of Handout |
| FileName | C12 | File containing contents of handout |
| | | to print |

Table 9 contains a list of letters, or patient notes, the practice uses. An example would be a work restriction that would be given to the patient.

TABLE 9
Letters Table

| | | |
|---|---|---|
| Code | C5 | Primary Key |
| Description | C60 | Name of Letter/Note |
| FileName | C12 | File containing text to print |

Table 10 contains a list of commonly given verbal instructions. These instructions are generally one or two sentences. They describe the information the doctor verbally gave to the patient. An example would be “Patient was reminded about the importance of using a seatbelt” The text of the instruction appears in the note for the visit.

TABLE 10
Instructions Table

| | | |
|---|---|---|
| Code | C5 | Primary Key |
| Description | C60 | Name of Instruction |
| FileName | C12 | File containing text to insert in note |

Table 11 contains the text of detailed, problem-specific reminders that appear in a pop-up window to a doctor. Where present, it is the first thing a doctor sees after selecting a diagnosis. (See example of Ace Inhibitor for Diabetic Care above).

TABLE 11
Note/Alert Table

| | | |
|---|---|---|
| Code | C5 | Primary Key |
| Description | C60 | Name of Note/Alert |
| FileName | C12 | File containing text to display |

Table 12 contains information regarding specialties. Since referrals to specialists are linked to a specialty type, system 10 is then able to provide a selection window of all of the specialists that appear in the following table of the specified specialty type. An example would be Podiatry.

TABLE 12
Specialty Type Table

| | | |
|---|---|---|
| Code | C3 | Primary Key |
| Description | C60 | Name of Specialty |

Table 13 contains a list of all specialists in the area with their respective specialties.

TABLE 13
Specialist Table

| | | |
|---|---|---|
| Code | C5 | Primary Key |
| SpecialtyCode | C3 | Links to Specialty Type Code |
| FirstName | C15 | |
| MI | C1 | |
| LastName | C15 | |
| Address1 | C25 | |
| Address2 | C25 | |
| City | C25 | |
| State | C25 | |
| Zip | C5 | |
| PhoneNo | C12 | |
| FaxNo | C12 | |

Table 14 contains a list of all of the medications the practice prescribes. These are most often keyed by a National Drug Code as established by Health and Human Services. The present invention may link to a specific medication, or, it may link to a class of medications. If it is linked to a medication class, the doctor is presented a window of all of the medications within the specified class from which to choose.

TABLE 14
Medication Table

| | | |
|---|---|---|
| Code | C11 | Primary Key (usually a NDC code) |
| ClassCode | C3 | Links to Medication Class Code |
| Description | C40 | Name of Medication |

Each medication is classified using the ClassCode to link to Tables 15 and 16. Table 15 contains a simple list of all of the medication classes, each with a code and description. Table 15 exists in a one-to-many relationship with Table 16. Table 16 contains one record for each medication in the class. It links the Table 15 to Table 14.

TABLE 15
Medication Class Table

| | | |
|---|---|---|
| Code | C5 | Primary Key |
| Description | C60 | Name of Medication Class |

TABLE 16
Medication Class List

| | | |
|---|---|---|
| ClassCode | C5 | Links to Medication Class Code |
| RxCode | C11 | Links to Medication Table Code |

Table 17 shows the basic data structure for the system 10's data entry forms, used by the text generation engine of the system to generate documentation.

TABLE 17
Form Table

| | | |
|---|---|---|
| Code | C5 | Primary Key |
| Description | C60 | Name of Data Entry Form |

Each data entry form has a code and a description. The form contents (the fields contained within the form) are stored in Table 18.

TABLE 18
Form Detail

| | | |
|---|---|---|
| FormCode | C5 | Links to Form Code |
| FieldNo | C5 | Unique Identifier for each field on |
| | | form |
| Description | C40 | Name of Field |
| FieldType | C5 | Indicates type of data (e.g., |
| | | checkbox, textbox, radio button, |
| | | drop-down selection, numeric, etc.) |
| PageNo | N1 | Indicates page this field appears on |
| PosX | N5 | Indicates horizontal position |
| PosY | N5 | Indicates vertical position |

Each form may have an unlimited amount of PreSets. A PreSet is a set of values. For example, every form has a Normal PreSet—this is a collection of all of the “normal” values of the fields contained in the form. PreSets are used to store the expected values of the fields on a form under certain circumstances. For example, there might be a PreSet called Hypertension of the Neck form that contains the most common values of the data on the Neck form when a patient presents with Hypertension. The use of PreSets requires Tables 19 and 20. Table 19 contains a simple list of PreSets by form, each with its own description. Every PreSet is related to a specific form. A form can have an unlimited number of PreSets.

TABLE 19
PreSet Table

| | | |
|---|---|---|
| Code | C5 | Primary Key |
| FormCode | C5 | Links to Form Code |
| Description | C60 | Name of PreSet |

Table 20 stores details of each PreSet.

TABLE 20
PreSet Detail

| | | |
|---|---|---|
| PresetCode | C5 | Links to PreSet Code |
| FormCode | C5 | Links to Form Code |
| FieldNo | C5 | Links to Form Detail |
| Value | V | Specific value for this Field on this |
| | | Form with this PreSet |

The purpose of Table 20 is to store specific values of each field in a form under the circumstances of the PreSet. E.g., the value of the Habitus field of the General Appearance form in the Normal PreSet might be well-nourished. Taking it one step further, then, whereas a PreSet is a collection of field values on an individual form, a Profile is a collection of PreSets. Thus a Profile is a list of selected forms, each with a selected PreSet. The Cardiopulmonary Exam Profile, for example, would contain a PreSet for each of the five forms selected to be a part of the Profile: General, Neck, Heart, Chest, and Extremities. Thus by selecting the Cardiopulmonary Exam Profile, hundreds of fields in the five data entry forms would have appropriate values set automatically as determined by the specific PreSets for each of the forms included in the Profile.

Tables 21 and 22 contains the table layout necessary to add this final dimension of documentation efficiency noted supra.

TABLE 21
Profile Table

| | | |
|---|---|---|
| Code | C5 | Primary Key |
| Description | C60 | Name of Profile |

TABLE 22
Profile Detail

| | | |
|---|---|---|
| ProfileCode | C5 | Links to Profile Code |
| FormCode | C5 | Links to Form Code |
| PreSetCode | C5 | Links to Preset Code |

Table 21 contains a simple list of profiles, each with an associated code. It is in a one-to-many relationship with the Profile Detail table listed below. Table 22 contains a specific PreSet for each of the forms included in the Profile.

Tables 18 through 22 enable system 10 to link a medical condition to a Profile. As a result, a very large number of data entry fields are populated with data that is appropriate for the medical condition presented as soon as the problem is inputted to system 10. The final result, of course, is the text generated in the note for the medical visit in question.

In real-time use, data from the library for the present invention causes a present invention window to appear when a diagnosis (problem or complaint) is indicated. This is accomplished in one of three ways: the MP indicates the patient is being seen for a follow-up visit on a chronic problem, for example, via a Problem List or similar such window in an EMRS (see FIG. 2); the MP selects a diagnosis from an Electronic Encounter or other such window in an EMRS (see FIG. 6); or the MP documents a chief complaint in an EMRS. In any of the above, a specific diagnosis code is internally selected by the EMRS. This triggers the present invention to open a window presenting the items associated with that code. For example, a user selected Hypercholesterolemia in FIG. 2, and system 10 presented window 110 in FIG. 3. Additionally, if that diagnosis code is a member of a family of diagnoses, system 10 also will present the elements pertinent to the entire family of diagnoses. Present invention windows are dynamically created based on the information contained in library 16.

Thus, it is seen that the objects of the invention are efficiently obtained, although changes and modifications to the invention should be readily apparent to those having ordinary skill in the art, without departing from the spirit or scope of the invention as claimed. Although the invention is described by reference to a specific preferred embodiment, it is clear that variations can be made without departing from the scope or spirit of the invention as claimed.

What is claimed is:

1. A method for processing work flow regarding a medical visit, comprising the steps of:
identifying the selection, in an electronic medical records system (EMRS), of a condition during said visit; and,
displaying a treatment plan regarding said condition, where said steps of identifying and displaying are performed by at least one general-purpose computer specially programmed to perform said steps of identifying and displaying.

2. The method recited in claim 1 wherein said selection is regarding treatment, documentation, or billing.

3. The method recited in claim 1 further comprising:
selecting said treatment plan from a library comprising a plurality of treatment plans, where said step of selecting is performed by said general-purpose computer.

4. The method recited in claim 3 further comprising:
at least partially populating said library from a first standard of treatment, where said step of populating is performed by said general-purpose computer; or,
accepting data from said EMRS and at least partially populating said library with said data, where said steps of populating from a first standard, accepting, and populating with said data are performed by said general-purpose computer.

5. The method recited in claim 1 further comprising:
accepting an editing input; and,
modifying said treatment plan in response to said editing input, where said steps of accepting and modifying are performed by said general-purpose computer.

6. The method recited in claim 1 wherein said treatment plan further comprises a first guideline; and,
said method further comprising:
designating said first guideline as a default guideline, where said step of designating is performed by said general-purpose computer.

7. The method recited in claim 6 further comprising:
accessing a second standard of treatment; and,
accepting an input from a user, said input regarding said first guideline, where said steps of accessing and accepting are performed by said general-purpose computer; and,
wherein designating said first guideline as a default guideline further comprises designating in accord with said input or in accord with said second standard of treatment.

8. The method recited in claim 1 wherein said treatment plan further comprises a second guideline and said second guideline is selected from the group consisting of procedure tasks, medication tasks, written materials tasks, documentation tasks, and practioner information tasks.

9. The method recited in claim 1 wherein said treatment plan further comprises a third guideline; and,
said method further comprising: executing said third guideline, where said step of executing is performed by said general-purpose computer.

10. The method recited in claim 9 wherein said third guideline is selected from the group consisting of sending information to other parties; generating medication prescriptions; creating letters regarding said patient; providing written materials for said patient; generating medical service orders; triggering procedures to be performed; and generating a history and documenting examination findings.

11. The method recited in claim 1 wherein said EMRS comprises at least one data entry form; and,
said method further comprising:
generating at least one code set value regarding said condition; and,
communicating said at least one code set value to said EMRS for use in prepopulating said at least one data entry form, where said steps of generating and communicating are performed by said general-purpose computer.

12. A computer-based system for processing work flow regarding a medical visit, comprising:
an interface element arranged to identify the selection, in an electronic medical records system (EMRS), of a condition during said visit; and,
a library comprising a plurality of treatment plans, where said library is arranged to select a treatment plan from said plurality of treatment plans in response to said identification, said interface element is arranged to output said treatment plan to said EMRS for display, and where said interface element and said library are included in at least one specially programmed general-purpose computer.

13. The system of claim 12 wherein said selection is regarding treatment, documentation, or billing.

14. The system of claim 12 further comprising:
a populating element arranged to at least partially populate said library from a first standard of treatment, or arranged to accept data from said EMRS and to at least partially populate said library with said data, said populating element included in said at least one specially programmed computer.

15. The system of claim 12 further comprising: an editing element; and,
wherein said interface element is arranged to accept an editing input and said editing element is arranged to modify said treatment plan in response to said editing input, and said editing element is included in said at least one specially programmed computer.

16. The system of claim 12 wherein said library has access to a second standard of treatment, said treatment plan further comprises a guideline, said interface element is arranged to accept input from a user, and said library is arranged to designate said guideline as a default guideline in accord with a second standard of treatment or in response to said input.

17. The system of claim 12 wherein said EMRS comprises at least one data entry form, said library is arranged to generate at least one code set value regarding said condition, and said interface element is arranged to output said at least one code set value to said EMRS for use by said EMRS in prepopulating said at least one data entry form.

18. A method for managing care guidelines in a network, comprising the steps of:
identifying the selection, in a first electronic medical records system (EMRS) in a first location in said network, of a first condition during a visit at said first location; and,
in response to said identifying said first condition, displaying, at said first location, a first treatment plan, from a plurality of treatment plans, regarding said first condition, where said steps of identifying and displaying are performed by at least one general-purpose computer specially programmed to perform said steps of identifying and displaying.

19. The method recited in claim 18 wherein said network comprises a second location with a second EMRS; and,
said method further comprising:
identifying the selection, in said second EMRS, of said first condition during a first visit at said second location; and,
displaying, at said second location, said first treatment plan, where said steps of identifying and displaying are performed by said general-purpose computer.

20. The method recited in claim 19 wherein said plurality of treatment plans further comprises a second treatment plan; and,
said method further comprising:
displaying, at said second location, said second treatment plan, where said step of displaying is performed by said general-purpose computer.

21. The method recited in claim 18 wherein said plurality of treatment plans further comprises a third treatment plan; and,
said method further comprising:
identifying the selection, in said second EMRS, of a second condition during a second visit at said second location; and,
displaying, at said second location, said third treatment plan, where said steps of identifying and displaying are performed by said general-purpose computer.

22. A computer-based system for managing care guidelines in a network, comprising:
an interface element arranged to identify the selection, in a first electronic medical records system (EMRS) in a first location in said network, of a first condition during a visit to said first location; and,
a library comprising a plurality of treatment plans, where said library is arranged to select a first treatment plan from said plurality of treatment plans in response to said identifying said first condition, said interface element is arranged to output said first treatment plan to said first location for display, and where said interface element and said library are included in at least one specially programmed general-purpose computer.

23. The system of claim 22 wherein said network comprises a second location with a second EMRS, said interface element is arranged to identify the selection, in said second EMRS, of said first condition during a first visit to said second location, said library is arranged to select said first treatment plan in response to said identifying said first condition, and said interface element is arranged to output said first treatment plan to said second location for display.

24. The system of claim 23 wherein said plurality of treatment plans comprises a second treatment plan, said library is arranged to select said second treatment plan in response to said identifying said first condition, and said interface element is arranged to output said second treatment plan to said second location for display.

25. The system of claim 23 wherein said plurality of treatment plans further comprises a third treatment plan, said interface element is arranged to identify the selection, in said second EMRS, of a second condition during a second visit to said second location, said library is arranged to select said third treatment plan in response to said identifying said second condition, and said interface element is arranged to output said third treatment plan to said second location for display.
